(19) United States
(12) Patent Application Publication
(10) Pub. No.: US 2016/0089548 A1
(43) Pub. Date: Mar. 31, 2016

(54) APPARATUS AND METHOD FOR PROMOTING D-VITAMIN PRODUCTION IN A LIVING ORGANISM

(71) Applicant: SR LIGHT APS, Herning (DK)

(72) Inventor: Povl Kaas, Herning (DK)

(21) Appl. No.: 14/891,587

(22) PCT Filed: May 15, 2014
(86) PCT No.: PCT/EP2014/059910

§ 371 Date: Nov. 16, 2015

(30) Foreign Application Priority Data
May 17, 2013 (EP) 13168238.7

(51) Int. Cl. A61N 5/06; F21V 29/70; F21K 99/00; A01K 29/00

(52) U.S. Cl. CPC A61N 5/0613; A61N 5/0625; A01K 29/00; F21V 29/70; F21K 9/30; A61N 2005/0626; A61N 2005/0652; A61N 2005/0659; A61N 2005/0661; F21Y 2101/02

(57) ABSTRACT

The invention regards an Apparatus for promoting D-vitamin production in a living organism, comprising at least one lamp assembly, said at least one lamp assembly is adapted to emit light, wherein the light at least emulates natural or IR light and UV light at wavelengths between 270 nm and 315 nm, wherein the at least one lamp assembly comprises a plurality of LEDs.

### FIELD OF INVENTION

The invention relates to an apparatus for promoting D-vitamin production in a living organism, comprising at least one lamp assembly. The invention further relates to the use of such apparatus and a method for increasing D-vitamin production in an animal.

### BACKGROUND OF THE INVENTION

It is known in the art that natural light promotes the production of D-vitamins in the human body.

In EP 1 970 423 A1 a fluorescent lamp for stimulating previtamin D3 production in the human skin is disclosed. The lamp is a low pressure mercury discharge lamp, which has a limited intensity and produces monochromatic light, i.e. just one dominant spectral line.

WO 2009/094100 discloses an enhanced UV-emitting fluorescent lamp that provides a UV spectral emission for simultaneously tanning of the human skin and promotion of vitamin D production in the human body. The disclosed lamp is a monochromatic low pressure lamp.

Low pressure lamps only supply monochromatic light (just one dominant spectral line). In addition the power rating does not go beyond a few hundred watt, which is a disadvantage if the lamps are to be installed, for example, in a cowshed at a distance of 3 to 4 meters from the cows.

WO2010/102039 discloses a method for increasing the vitamin D content of a mushroom by exposing the mushroom to a lamp that emits UVA and UVB, but not UVC radiation. It is noted that the mushrooms are only exposed to UVA and UVB radiation but not natural light. In addition, it is noted that there is an increase in the content of D2 vitamin and for example not of the other important D vitamins, such as the D3 vitamin.

It is known that when an animal, such as a cow, is kept outside in a field in the summer, where it is exposed to natural sunlight, its D-vitamin status is increased and the content of D-vitamin in its milk is accordingly increased. As modern farming involves keeping the livestock indoor, inside an animal farm production facility, the natural production of D-vitamin is compromised. To compensate, a D-vitamin supplement in the food is used. However, it is known that the D-vitamin level, obtainable by exposing a human or an animal to sunlight, is much higher than the level obtainable through food supplements. Furthermore, it has been discovered that the skin of a human or an animal is a much more efficient in producing D-vitamin if exposed to sunlight and it is not possible to achieve a toxic dozes of D-vitamin when exposed to the sun.

WO2013/041389 discloses an apparatus for promoting D-vitamin production in a living organism, comprising at least one lamp assembly, said at least one lamp assembly is adapted to emit polychromatic light, wherein the polychromatic light at least emulates natural light and UV light at wavelengths between 270 nm and 315 nm, wherein the at least one lamp assembly comprises at least one medium and/or high pressure lamp. When using a medium and/or high pressure lamp to generate UV light a substantial amount of heat is generated and it uses a lot of power. In addition, is it difficult to precisely generate the desired output spectrum and intensity of the UV light.

Considering the prior art described above, it is an object of the present invention to provide an apparatus and method for promoting D-vitamin production, in a living organism in an efficient, cost effective and reliable way. In particular, it is an object to increase the production of D3 vitamin in a livestock, such as cattle, pigs, chickens and the like.

### SUMMARY OF THE INVENTION

The object is achieved by an apparatus for promoting D-vitamin production in a living organism, comprising at least one lamp assembly, said at least one lamp assembly is adapted to emit light, wherein the light at least emulates natural or IR light and UV light at wavelengths between 270 nm and 315 nm, wherein the at least one lamp assembly comprises a plurality of LEDs.

By providing a combination of natural light and UV light at wavelengths between 270 nm and 315 nm, the production of D-vitamin in living organisms is enhanced. In relation to the dairy farming industry, a specific advantage is that in milk producing mammals, such as cows, the

D-vitamin content of the milk is increased. It has been found, that the vitamin D3 content is increased, especially the D-vitamin content in milk from cows is increased when exposed to light comprising both natural light and UV light at wavelengths between 270 nm and 315 nm.

By providing a combination of IR light and UV light at wavelengths between 270 nm and 315 nm, the production of D-vitamin in living organisms can be enhanced.

By the use of a plurality of light-emitting diodes (LED), it is possible to get the desired light profile. A LED is a semiconductor light source that emits monochromatic light. By using a plurality of different LEDs it is possible to build the desired light profile having the desired intensity and wavelengths. Further, the use of LED's has the advantage that is uses less power than other light sources, such as medium and/or high pressure lamps.

A LED does not generate the same amount of heat as a conventional light source and can therefore be positioned closer to the living organism without the risk of heating and/or burning it.

Natural light is to be understood as sunlight as it appears on the earth surface during the day within the normal variations in intensity and spectrum. The spectrum and intensity of sunlight varies depending on the atmosphere, ozone layer and the position of the sun, and here natural light refer to any sunlight within those normal variations.

Although some LEDs are considered to have only one wavelength, it is known to the skilled person that a LED has a narrow wavelength distribution around a central wavelength.

In an embodiment, the plurality of LEDs are adapted to generate the UV light at wavelengths between 270 nm and 315 nm. Preferably, a plurality of LEDs provide a plurality of wavelengths within the range from 270 nm to 315 nm. Prior art lamps normally use means for removing light of a wavelength under 270 nm. Wavelengths under 270 nm are undesired as it involves a health risk to expose living organisms to it. Using LEDs to generate the UV light makes the preferred embodiment simpler, as there is no need for a filter. The means are usually a filter that is transparent to light of wavelengths above 270 and opaque for wavelengths under 270 nm.

In an embodiment, the light appears to be continuous, such as light at a frequency over 50 Hz. This can be an advantage if for example a discharge lamp is used for generating the light that emulates natural light. The use of a flashing or pulsing light will stress the living organism. Therefore, it is an advantage to ensure that the light appears to be continuous to the living organism, such as an animal. This can be ensured by having a frequency above 50 Hz, 100 Hz, 200 Hz, 500 Hz, 1000 Hz, 2000 Hz or 3000 Hz.

In an embodiment, the at least one lamp assembly comprises at least one first lamp emulating natural light and the plurality of LEDs provides UV light at wavelengths between 270 nm and 315 nm. In this embodiment, the first lamp can be any lamp that emulates natural light, such as a sulphur lamp, and the second lamp can comprise the plurality of LEDs which emits light at wavelength between 270 nm and 315 nm. There can be any number of lamps in order to secure that the area covered by the emitted light is as intensive as possible.

In an embodiment, at least some of the plurality of LEDs are mounted on a plate. This embodiment enables the use of only one lamp having a plurality of LEDs providing the full spectrum needed.

Preferably, the plate is heat conducting and/or has a temperature sensor attached for the determination of the temperature of the LEDs mounted on the plate, preferably the apparatus further comprises means for controlling the temperature of the plate. The properties of a LED is dependent on the temperature. For example, the wavelength can change and the efficiency can be higher at lower temperature. Therefore, it is preferred that all the LEDs behave in the same way this is achieved by the use of a heat conducting plate in thermodynamic contact with the LEDs. Further, it is preferred to control the temperature; for example by means of a liquid cooling circuit in thermodynamic contact with the plate and/or a heat sink which can remove heat from the LEDs.

Advantageously, the apparatus further comprises a lens in front of the plurality of LEDs. The lens can be part of each individual LED, cover a number of or all the LEDs. The lens can be used to focus the light hereby making it possible for the light emitting from a lamp to be directed at a specific area.

A further aspect of the present invention is the use of an apparatus as described herein to increase D-vitamin production in animals. The apparatus as described above can be used to enhance the D-vitamin production within the living organism of an animal. Thus, the D-vitamin content in the meat from the animals exposed to the apparatus described above is increased. This can prevent humans eating meat from cows suffering from vitamin D deficiency. These animals can be birds, such as chickens and/or non-human mammals, such as cows, pigs, goats and/or lambs.

The invention also concerns the use of an apparatus as described to increase D-vitamin content in milk from a non-human mammal, preferably a cow. Increasing the vitamin D content in milk by using of the present invention has the advantage that humans consuming the milk can prevent vitamin D deficiency. In parts of the earth away from equator, the daytime light can be limited during winter. This, for example, is the case in northern Europe during the months November to March. During that time, the lack of exposure to the sun can cause people to suffer from vitamin D deficiency. In any case the sun is only important if the cows are outside. In Northern Europe, and other industrialised countries at the same latitude north or south, the majority of milk cows are inside a cowshed and as a consequence do not produce D-vitamins in their milk unless the animals are exposed to the light produced by an apparatus according to the first aspect of the present invention. Thus, by the present invention, a compensation for the lack of natural sunlight is provided, whereby it is possible to produce milk during the winter, naturally enhanced with D-vitamin and thereby help preventing the lack of D-vitamin in the human body of the people drinking the milk. This is particularly advantageous in relation to organic farming where artificial nutritious food supplements are not allowed. By the present invention, the D-vitamin content is kept at a high level without any dietary supplements to the animal food whereby the animals are farmed organically.

Another aspect of the invention concerns the use of an apparatus as described above to expose at least one animal to light produced by an apparatus according to the first aspect of the present invention for a predetermined amount of time daily in order to increase the content of D-vitamin in the milk produced by said at least one animal. Hereby the production of D-vitamin can be optimised. For example, the animals can be exposed to light from the apparatus according to the invention for 30 minutes daily, 60 minutes daily, 90 minutes daily or 120 minutes daily.

Another aspect of the present invention concerns the use of an apparatus as described above wherein the light at least emulates IR light and that the IR light is used to keep the animals warm. IR light is conventionally used to warm suckling pig, piglets and chickens. Adding UV light will ensure that the suckling pig, piglets or chickens do not lack of shortage of D-vitamin and the D-vitamin content in the meat. This can prevent humans eating meat from cows suffering from vitamin D deficiency. When keeping the suckling pig, piglets or chickens warm care should be taken not to heat too much. Therefore it is an advantage to use LEDs to provide the UV light it provides no change in the heating properties of the lamp. The LEDs providing the UV light can be positioned or attached to the conventional IR light source hereby providing a compact unit that can be installed without modification of a conventional accommodation for the suckling pig, piglets or chickens.

An additional aspect of the present invention is an animal farm production facility comprising at least one apparatus according to the present invention. As the apparatus emits both visible light and UV light, an animal farm production facility with the apparatus of the present invention does not need any additional lightening system. The present invention can substitute the conventional lighting of the cowshed, which saves money at installation as only one illumination system is needed.

In accordance with this aspect, an animal farm production facility, wherein one or more livestock are accommodated for production of one or more farm products, might apply the lighting apparatus throughout the entire building or it could be one or more lamps in the ceiling lighting amongst traditional lighting facilities. According to the invention, the animal farm production facility could also include one or more cows which are accommodated with the aim to produce milk, where the light source is provided in relation to a milking station or the like. Preferably, one or more cows are accommodated for production of milk and/or one or more birds, such as chickens or hens, are accommodated for production of eggs and/or one or more pigs are accommodated for production of meat.

In general, it is realised that the effect of increasing the D-vitamin will benefit the health of the livestock. The animals will thereby achieve a better immune system, whereby less or no antibiotics is needed and less environmental impact, just as a natural D-vitamin source is provided for humans when utilising the invention in an animal farm production.

By the invention, it is also realised that the using the apparatus in an animal farm production is beneficial to birds, such as chickens or hens, for production of eggs. The promotion of D-vitamin in the eggs increases the nutritional value of the eggs as a human food source.

In an embodiment, the light is provided by a plurality of lamps. This can make the installation easier and in addition it is possible to turn off only some of the lamps. This can be advantageous, if for example a first lamp of natural light and a second lamp of the UV light at wavelengths between 270 nm and 315 nm are used. Then the UV light having wavelengths between 270 nm and 315 nm can be turned on for only a limited time for example 2 to 3 hours every day and the natural light can be on the entire day and be used as the primary illumination of the building.

### DESCRIPTION OF THE DRAWINGS

The invention will in the following be described in greater detail with reference to the accompanying drawings, in which:

FIGS. 1A and 1B shows a schematic side and front view—respectively—of a lamp assembly according to an embodiment of the present invention;

FIGS. 2A and 2B shows a schematic side and front view—respectively—of a lamp assembly according to an embodiment of the present invention;

FIGS. 3A and 3B shows a schematic side and front view—respectively—of a lamp assembly according to an embodiment of the present invention;

FIGS. 4A and 4B shows a schematic side and front view—respectively—of a lamp assembly according to an embodiment of the present invention;

FIGS. 5A and 5B shows a schematic side and front view—respectively—of a lamp assembly according to an embodiment of the present invention;

FIG. 6 shows a graph showing test results;

FIG. 7 shows a comparative test of D3 vitamin contents in pigs; and

FIG. 8 shows a comparative test between natural exposure and exposure to lightning from a lamp according to the invention.

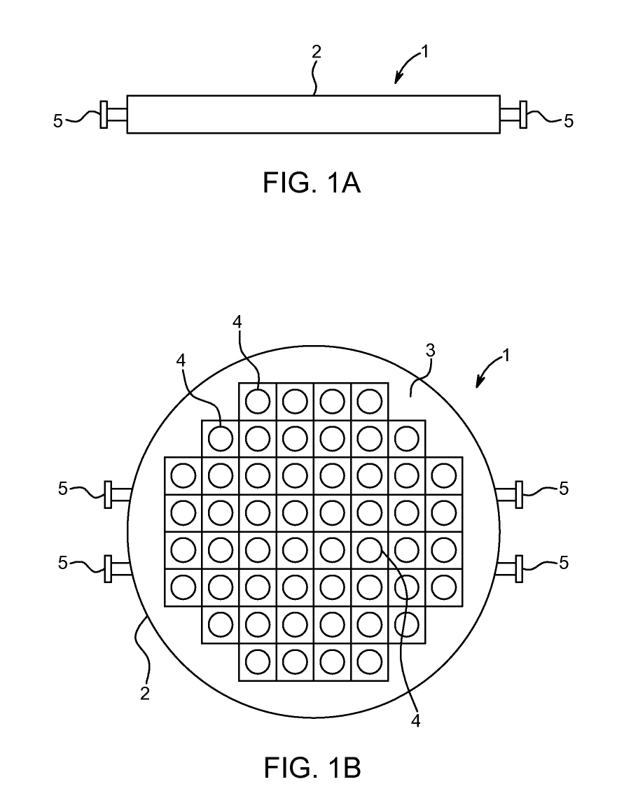

FIGS. 1A and 1B shows an embodiment of a lamp 1 comprising a housing 2 with a heat conducting plate 3 on which LEDs 4 are attached. The lamp 1 has a circular form. FIG. 1A is a front view of the lamp 1 and 1B is a side view of the lamp 1. In this embodiment there are 52 LEDs 4. The lamp 1 can both be adapted to provide the desired spectrum of UV light and/or natural light. It is to be understood that the embodiment shown if FIG. 1 can be adapted is such a way that more LEDs 4 are incorporated in to the lamp, for example the lamp can have in excess of 100 LEDs 4. The LEDs 4 emits light with a wavelength narrow distributed around a central wavelength. When using a plurality of LEDs it is possible to build a lamp with the desired profile; the specific desired wavelengths can be chosen and the intensity for a specific wavelength can be enhanced by using several of the same LEDs. In this way the lamp 1 can be built to provide the desired spectrum.

The heat conducting plate 3 is in thermodynamic contact with the LEDs 4 and can be cooled by use of a fan in order to hinder that the LEDs 4 overheats. The lamp 1 is connected to a power supply by use of the connectors 5, hereby providing power to the LEDs 4.

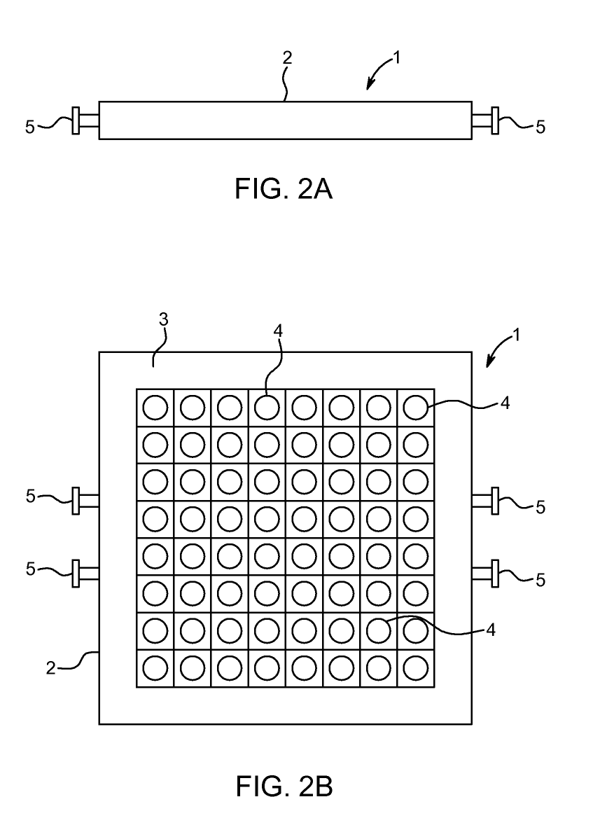

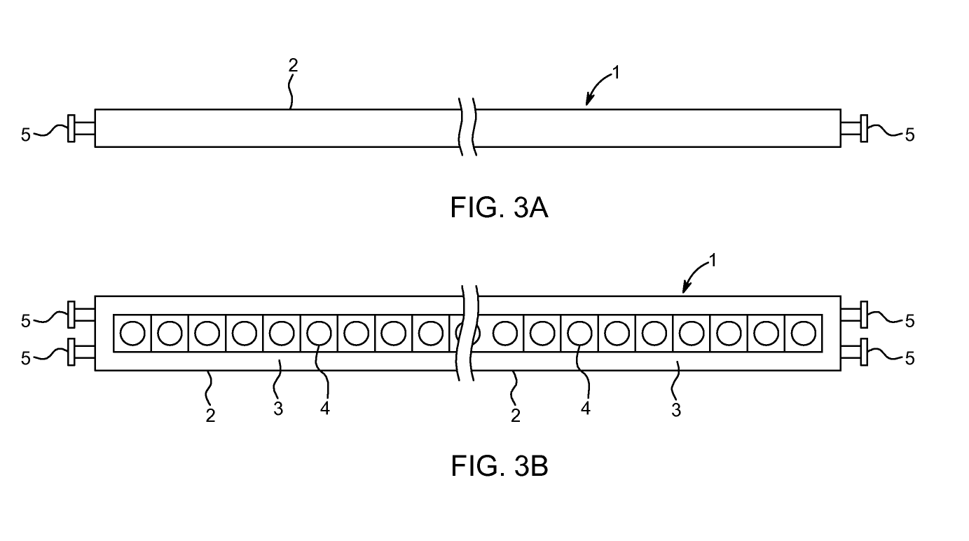

The lamps on FIGS. 2A and 2B, and 3A and 3B discloses a lamp 1 similar to the lamp disclosed in FIGS. 1A and 1B. However, the lamp in FIG. 3 has a square form and the lamp in FIG. 3 has the LEDs arranged in a row. It is to be understood that the lamp 1 shown in FIGS. 3A and 3B can have any length and only the ends of the lamp 1 are shown on the FIG.

In FIGS. 3A and 3B a lamp similar to the lamps of FIGS. 1, 2 and 3 is shown, however the housing has a different form.

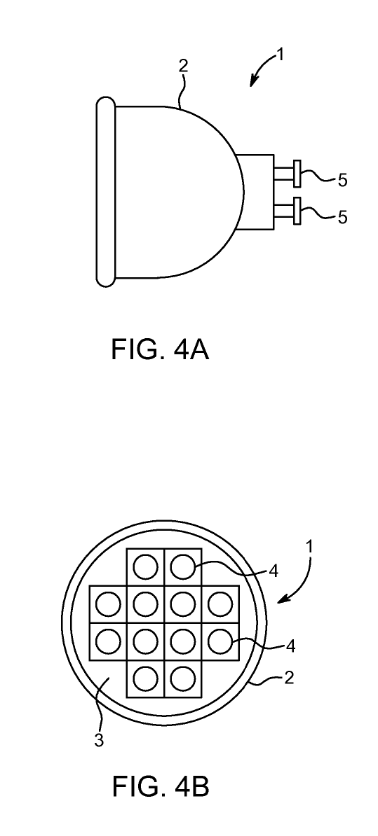

The lamp assembly shown on FIGS. 3 and 4 are especially suitable for battery chicken or other animals in cages, because those lamp assemblies are suitable in size to be positioned on or very close to the chickens or animals. The lamp assembly in FIG. 3 can be made to cover a whole row of cages. Each cage can also have a lamp assembly as the one shown in FIG. 4 attached.

The lamp assembly can be made so that one LED lamp provides the UV light with wavelength between 270 nm and 315 nm and another LED lamp provides the natural light. However, a sulphur and/or mercury lamp can also be used to provide natural light as the spectrum of a sulphur lamp is very similar to daytime solar light at the earth's surface. A sulphur and mercury lamp can be doped with metals in order to achieve light emission as close to natural light as technically possible. Sulphur and mercury lamps are known in the art and will not be described in detail.

If the lamp assembly comprises two lamps, one emitting natural light and the other UV light, it is possible to regulate the UV and the natural light independently. As an example, such a lamp assembly can be installed in an animal farm production building, the natural light lamp can be used to light the inside of an animal farm production building with light similar to daylight, enabling the farmer to work in the animal farm production facility, such as a building accommodating cows. The UV lamp can then be turned on and off for only a limited amount of time each day, for example 1, 2 or 4 hours every day. By using the natural light lamp (here exemplified as a LED, mercury and/or sulphur lamp) as lighting in the building, no other lighting installation is needed. Hence, the costs of the usual lighting can be reduced.

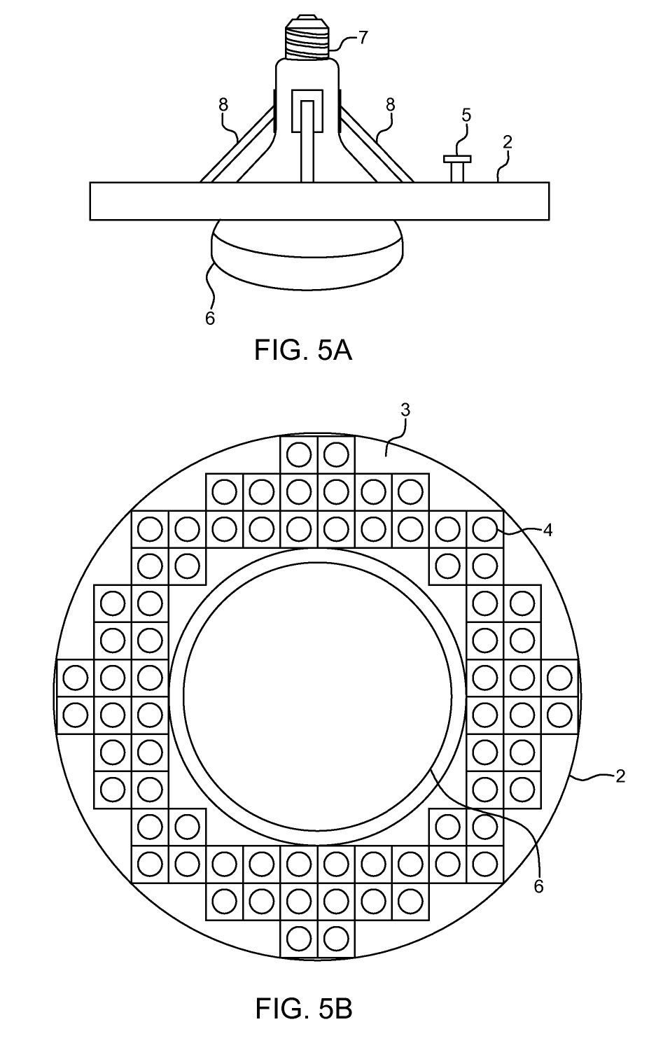

FIG. 5 shows a lamp 1 comprising a IR-light bulb 6 of the type used for heating. It can, for example, be the type used for keeping suckling pig, piglets or chickens warm. The IR-light bulb 6 has a plug 7 for connection to a conventional socket. A UV lamp housing 2 is attached to the IR-light bulb 6 by use of the rods 8. The UV lamp housing 2 comprises LEDs 4 adapted to provide UV light at wavelengths between 270 nm and 315 nm. The IR-light bulb 6 is usually positioned very close to the suckling pig, piglets or chickens it is therefore an advantage that the UV lamp utilises LEDs as this will prevent the lamp 1 from getting too warm for the suckling pig, piglets or chickens. The conducting plate 3 can be thermodynamically connected to means for controlling the temperature, so that the temperature can be kept constant and any heat from the IR-light bulb does not effect the LEDs performance.

In the case where the LED lamp provides both the natural light and the UV light, a farmer only needs to install this type of lamp in his livestock accommodating production facilities in order to increase the vitamin D content of the animal product.

The following table includes examples of different lamp types and single LEDs that can be used in a lamp assembly.

| | | Min. | Max. |
|---|---|---|---|
| Lamp type | no. | nm | nm |
| UV-LED wavelength ±2% | 1 | 280 | 280 |
| UV-LED wavelength ±2% | 2 | 285 | 285 |
| UV-LED wavelength ±2% | 3 | 290 | 290 |
| UV-LED wavelength ±2% | 4 | 295 | 295 |
| UV-LED wavelength ±2% | 5 | 297 | 297 |
| UV-LED wavelength ±2% | 6 | 300 | 300 |
| UV-LED wavelength ±2% | 7 | 303 | 303 |
| UV-LED wavelength ±2% | 8 | 305 | 305 |
| UV-LED wavelength ±2% | 9 | 310 | 310 |
| UV-LED wavelength ±2% | 10 | 315 | 315 |
| UV-LED wavelength ±2% | 11 | 320 | 320 |
| UV-LED wavelength ±2% | 12 | 330 | 330 |
| LED visible light | 13 | 400 | 700 |
| Mercury florescent low pressure visible light | 14 | 380 | 750 |
| Mercury medium pressure visible light | 15 | 270 | 900 |
| Mercury high pressure visible light | 16 | 270 | 900 |
| Sodium low pressure visible light | 17 | 589 | 589.6 |
| Sodium high pressure visible light | 18 | 350 | 750 |
| Xenon high pressure visible light | 19 | 270 | 900 |
| Mercury florescent low pressure | 20 | 270 | 330 |
| Mercury medium pressure doped | 21 | 270 | 315 |
| Mercury high pressure doped | 22 | 270 | 900 |
| Xenon high pressure doped | 23 | 270 | 900 |
| Sulphur | 24 | 380 | 900 |
| Plasma and/or incandescent lamp. (Solar Simulator) | 25 | 380 | 900 |
| Doped Plasma and/or doped incandescent lamp; (Solar | 26 | 270 | 900 |
| Simulator) | | | |

With use of examples of the lamps in the table, different embodiments of a lamp assembly can be made.

The following table has examples of lamp assemblies made from the lamp types from the table shown above.

| Lamp and combinations of lamps | no. | no. |
|---|---|---|
| Lamp assembly A | 13 | 1, 2, 3, 4, 5, 6, 7, 8, 9, 10, 12 |
| Lamp assembly B | 13 | 2, 3, 4, 5, 6, 7, 8 |
| Lamp assembly C | 13 | 20 |
| Lamp assembly D | 13 | 21 |
| Lamp assembly E | 13 | 22 |
| Lamp assembly F | 13 | 23 |
| Lamp assembly G | 13 | 26 |
| Lamp assembly H | 14 | 1, 2, 3, 4, 5, 6, 7, 8, 9, 10, 12 |
| Lamp assembly I | 14 | 2, 3, 4, 5, 6, 7, 8 |
| Lamp assembly J | 15 | 1, 2, 3, 4, 5, 6, 7, 8, 9, 10, 12 |
| Lamp assembly K | 15 | 2, 3, 4, 5, 6, 7, 8 |
| Lamp assembly L | 16 | 1, 2, 3, 4, 5, 6, 7, 8, 9, 10, 12 |
| Lamp assembly M | 16 | 2, 3, 4, 5, 6, 7, 8 |
| Lamp assembly N | 17 | 1, 2, 3, 4, 5, 6, 7, 8, 9, 10, 12 |
| Lamp assembly O | 17 | 2, 3, 4, 5, 6, 7, 8 |
| Lamp assembly P | 18 | 1, 2, 3, 4, 5, 6, 7, 8, 9, 10, 12 |
| Lamp assembly Q | 18 | 2, 3, 4, 5, 6, 7, 8 |
| Lamp assembly R | 19 | 1, 2, 3, 4, 5, 6, 7, 8, 9, 10, 12 |
| Lamp assembly S | 19 | 2, 3, 4, 5, 6, 7, 8 |
| Lamp assembly T | 24 | 1, 2, 3, 4, 5, 6, 7, 8, 9, 10, 12 |
| Lamp assembly U | 24 | 2, 3, 4, 5, 6, 7, 8 |
| Lamp assembly V | 25 | 1, 2, 3, 4, 5, 6, 7, 8, 9, 10, 12 |
| Lamp assembly X | 25 | 2, 3, 4, 5, 6, 7, 8 |

Thus, the lamp assemblies A to B comprise only LEDs. The lamp assemblies C to G comprise LEDs for providing the natural light and another lamp for providing the UV light. The lamp assemblies H to X comprise a plurality of LEDs for providing the UV light and another lamp for providing the natural light.

As the lamp assembly can be used as lighting, it is preferred that the lighting appears to be continuous. A pulsed lamp will not only stress the animals but also make it difficult for the farmer to work, most of the known LED and natural light lamps are to some extend pulsed but as long as the pulses are higher than 50 Hz it will appear to both animals and humans to be continuous.

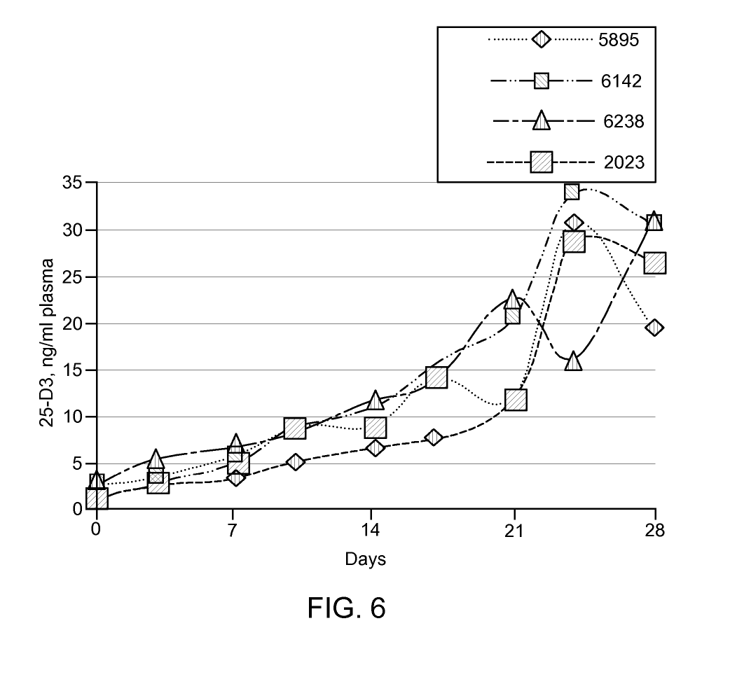

To disclose the effect on the milk produced by cows exposed to a lamp having the properties of the present invention, a test has been performed. Here, a UV lamp was used together with a lamp that emulated natural light to illuminate cows. The lamp assembly was installed 3 to 3.5 meters from the cows in the test. The lamp was turned on for 30 minutes every 24 hours. This was repeated for 28 days. The test was performed on four cows, having the numbers: 5895, 6142, 6238 and 2023. The cows were milked every day and the vitamin D3 content of the milk was measured for each of the four cows. The result of the test is shown in FIG. 6. It can be seen that the content of D3 vitamin increases from about 3 ng/ml to about 25 ng/ml. This is a substantial increase in the D vitamin content of the milk.

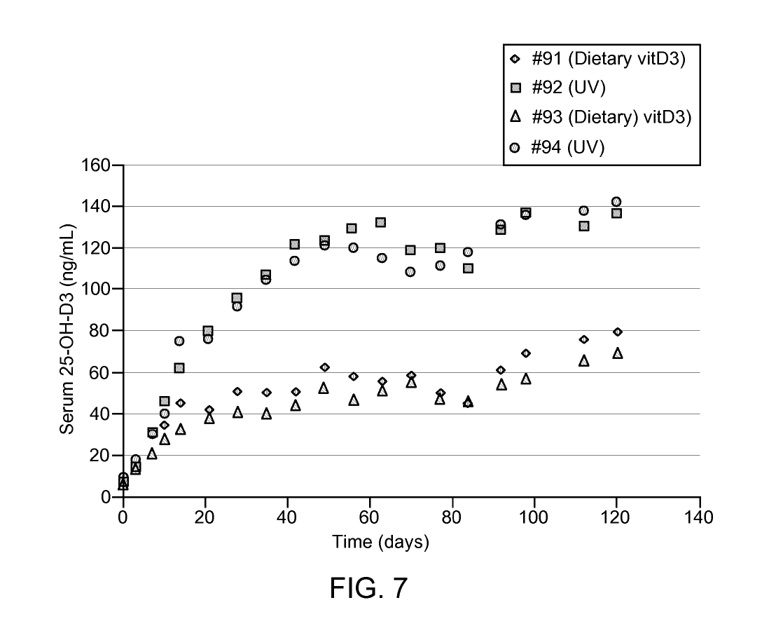

Besides increasing the content of D3 vitamin in the produced milk, it is realised that the UV lamp arrangement, according to the invention, may also increase the D-vitamin content in other animals. As shown in FIG. 7, a study of D vitamin status in pigs clearly shows an increase in the content of D-vitamin when the pigs are exposed to the UV lamp arrangement according to the invention (lots #92 and #94), compared to the animals given D3 vitamin in their food (lots #91 and #93).

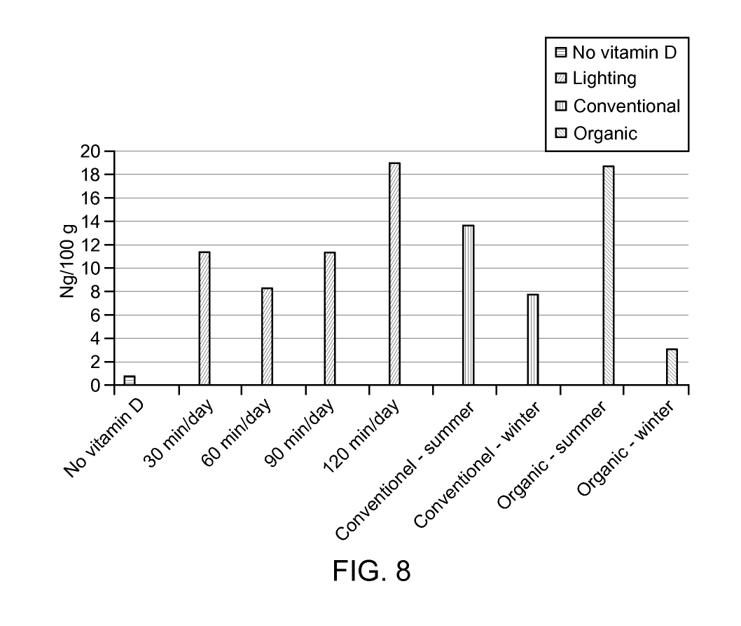

FIG. 8 shows the D3 vitamin content in the produced milk of cows—both conventional cattle and organic bread cattle—when exposed to natural sunlight during the winter and during the summer in comparison with an exposure to the lighting of the UV lamp arrangement according to the invention, for a predetermined amount of time every day. As shown in FIG. 8, studies also reveal that it is sufficient to expose the animals, such as cows, for 30 minutes every day in order to achieve the desired result to reproduce the D3 vitamin content, naturally occurring during summer when the animals are free-ranging outside and thereby exposed to sunlight. In particular, these studies reveal that by the present invention, it is possible to reproduce the same high amount of D3 vitamin content in milk as in the milk from organically farmed cows during summer, but all around the year.

1. Apparatus for promoting D-vitamin production in a living organism, comprising:
at least one lamp assembly, said at least one lamp assembly adapted to emit light;
wherein the light at least emulates natural or IR light and UV light at wavelengths between 270 nm and 315 nm; and
wherein the at least one lamp assembly comprises a plurality of LEDs.

2. Apparatus according to claim 1, wherein the plurality of LEDs are adapted to generate the UV light at wavelengths between 270 nm and 315 nm.

3. Apparatus according to claim 1, wherein the light appears to be continuous and has a frequency over 50 Hz.

4. Apparatus according to claim 1, wherein the at least one lamp assembly comprises at least one first lamp emulating natural light and the plurality of LEDs provide UV light at wavelengths between 270 nm and 315 nm.

5. Apparatus according to claim 1, further comprising a plate, wherein at least some of the plurality of LEDs are mounted on a plate.

6. Apparatus according to claim 5, wherein the plate is heat conducting and/or has a temperature sensor attached for the determination of the temperature of the LEDs mounted on the plate.

7. Apparatus according to claim 1, wherein the apparatus further comprises a lens disposed in front of the plurality of LEDs.

8-16. (canceled)

17. Apparatus according to claim 6, wherein the apparatus further comprises a temperature controller for controlling the temperature of the plate.

18. A method of increasing vitamin D production in an animal, comprising exposing the animal to the apparatus of claim 1.

19. A method according to claim 18, wherein the animal is a cow and the increasing vitamin D production increases the vitamin D content in milk from the cow.

20. A method according to claim 19, wherein the cow is exposed to light for a predetermined amount of time daily.

21. A method according to claim 18, wherein the light at least emulates IR light and that the IR light is used to keep the animals warm.
